(19) United States
(12) Patent Application Publication
(10) Pub. No.: US 2015/0164673 A1
(43) Pub. Date: Jun. 18, 2015

(54) Leg Brace and Walking Aid with Retractable Wheels

(71) Applicant: Thomas Rhodes, Springfeld, IL (US)

(72) Inventor: Thomas Rhodes, Springfeld, IL (US)

(21) Appl. No.: 14/572,145

(22) Filed: Dec. 16, 2014

Related U.S. Application Data
(60) Provisional application No. 61/916,401, filed on Dec. 16, 2013.

(51) Int. Cl. A61F 5/01

(52) U.S. Cl. CPC A61F 5/0127

(57) ABSTRACT

Provided is a leg brace device for restricting movement of the calf and foot areas of an injured leg. Some embodiments of the device may have an upright support connected to a foot support to form an L-shaped body. Straps extending across and/or around the body of the device may be employed to strap the calf and foot of a user to the body. Retractable wheels extend away from the exterior surface of the foot support and may be retracted into wheel recesses within the foot support. When deployed, the wheels are adapted to bear the weight of a user's leg during ambulatory motion. In this manner, the leg brace provides a means for rolling an injured limb.

### CROSS REFERENCE TO RELATED APPLICATION

This application claims the benefit of U.S. Provisional Application No. 61/916,401 filed on Dec. 16, 2013 entitled “Cast Mobility.” The above-identified patent application is herein incorporated by reference in its entirety to provide continuity of disclosure.

### BACKGROUND OF THE INVENTION

1. Field of the Invention

The present invention relates to walking aids. More specifically, it relates to a leg brace having retractable wheels that protrude from the sole of the foot covering when in use. The device removably affixes to a user's leg to provide support and enable rolling movement thereof.

Traumatic injuries to the leg or foot often require rigid support for an extended period of time while the limb heals. Injuries such as sprains and muscle tears may require compression support, while bone breaks may require a full cast to keep the limb immobile. Lower limb supports come in a variety of styles including bandage wraps, polyethylene compression coverings, air cushioned boots, and the like. The purpose of each of these devices is to hold the injured area in a maintained position while it heals, thus reducing the likelihood that muscles, bones, or ligaments will shift and exacerbate the injury.

Leg support devices are often cumbersome and heavy, rendering the affected leg difficult to move. Casts and splints may force users to walk while maintaining heir leg in an extended straight position. Patients are forced to hobble in order to move the extended limb forward. Crutches may be used to alleviate the difficulty of moving the limb, by enabling a patient to “walk” on the crutches and the healthy foot. This technique removes the pressure from the injured limb and may reduce some of the difficulty associated with moving it. Unfortunately, crutches are not an option for all patients, and some patients find it difficult to maneuver the injured limb despite having the crutches for support.

A lower limb support is needed that restricts the position of the leg and foot, while aiding in movement thereof. The present invention provides a supportive brace with retractable wheels disposed along the bottom of the sole. It thus supports the users injured leg or foot while aiding in improved mobility.

2. Description of the Prior Art

Devices have been disclosed in the prior art that relate to foot coverings with wheels. These include devices that have been patented and published in patent application publications. These devices generally relate to walking aids. The following is a list of devices deemed most relevant to the present disclosure, which are herein described for the purposes of highlighting and differentiating the unique aspects of the present invention, and further highlighting the drawbacks existing in the prior art.

DePalma, U.S. Pat. No. 5,832,560 discloses a protective foot and leg covering. The foot covering is a fully enclosing cast with a wheel disposed at the toe, where a toe stop lies on an ice skate. The bottom surface of the foot covering is coated with a high traction material that provides stability for the user when the sole of the foot is placed upon the ground. Unlike the present invention, the DePalma boot does not have retractable wheels on the sole of the foot covering and further teaches away from the notion of wheels disposed on the underside of the foot covering.

Schreyer et al, U.S. Pat. No. 3,983,643 discloses a shoe that can be utilized for walking or roller-skating. The shoe features two retractable wheels located on the underside of the shoe, which can be deployed according to the user's desire to walk or roller sate. The shoe does not provide leg or ankle support and does not extend up the leg of a user. The present invention provides an L-shaped leg support that aids in protecting an injured leg from further trauma. A similar shoe is disclosed in Adams, U.S. Pat. No. 8,480,095. The Adams shoe features a pair of retractable wheels located under the sole and near the heel. The primary purpose of the Adams device is for entertainment and sport, and thus does not contemplate protective support for the legs of users. Other examples of skate shoes can be found in Wilborn, U.S. Pat. No. 7,735,840 and Maxwell et al, U.S. Pat. No. 7,036,829.

A roller skate attachment for a foot or shoe is discloses in Ferguson, U.S. Pat. No. 2,484,494. The attachment has an elongated base that covers the sole of a shoe, and has several straps that cross over the top of the shoe and connect to an opposing side of the base. Wheels protrude outward from the base along the front and sides, allowing the user to roll forward along the sole of the foot, or on the toe area. This device does not provide retractable wheels, nor does it disclose a brace support for the leg of the wearer.

Olexenhko, U.S. Pat. No. 7,959,545 discloses another walking aid with wheels. The device provides a pedestal with a platform along the top and wheels disposed along the bottom. The shin of an injured leg is placed on the platform, such that the user's knee is bent underneath him or her. The supporting leg is free to walk and the injured leg rolls along on the wheels of the pedestal. Unlike the present invention, the Olexenhko device does not secure to a user's calf to provide a brace, nor does it have retractable wheels.

These prior art devices have several known drawbacks. They do not provide a leg brace that supports an injured leg, while providing the option of standing flatfooted, or rolling along the ground. The present invention provides people recovering rom leg injuries with a brace with retractable wheels, thereby enabling them to walk or roll the leg. It substantially diverges in design elements from the prior art and consequently it is clear that there is a need in the art for an improvement to existing walking aid devices. In this regard the instant invention substantially fulfills these needs.

### SUMMARY OF THE INVENTION

In view of the foregoing disadvantages inherent in the known types of walking aids now present in the prior art, the present invention provides a new retractable wheel set wherein the same can be utilized for providing convenience for the user when improving mobility of an injured leg or foot.

It is therefore an object of the present invention to provide a new and improved leg brace device that has all of the advantages of the prior art and none of the disadvantages.

It is another object of the present invention to provide a leg brace device that has retractable wheels, thereby providing a user with dual modes of ambulatory motion. A first mode with the wheels deployed to facilitate rolling of the injured limb. A second mode wherein the wheels are retracted to permit the user to stand on the braced limb,.

Another object of the present invention is to provide a leg brace adapted to restrict the motion of an injured leg.

Yet another object of the present invention is to provide a leg brace device capable of rolling along he ground.

A further object of the present invention is to provide a leg brace that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 illustrates a front view of an injured user employing a leg brace device according to an embodiment.

FIG. 2 illustrates a side view of an embodiment of a leg brace device secured to the calf of a user's leg.

FIG. 3 illustrates a side view of the footrest and wheels of a leg brace according to an embodiment.

FIG. 4 illustrates a side perspective view of an embodiment of the footrest and back support portions of a leg brace device.

### DETAILED DESCRIPTION OF THE INVENTION

Reference is made herein to the attached drawings. Like reference numerals are used throughout the drawings to depict like or similar elements of the leg brace device. For the purposes of presenting a brief and clear description of the present invention, the preferred embodiment will be discussed as used for supporting an injured limb during ambulatory motion. The figures are intended for representative purposes only and should not be considered to be limiting in any respect.

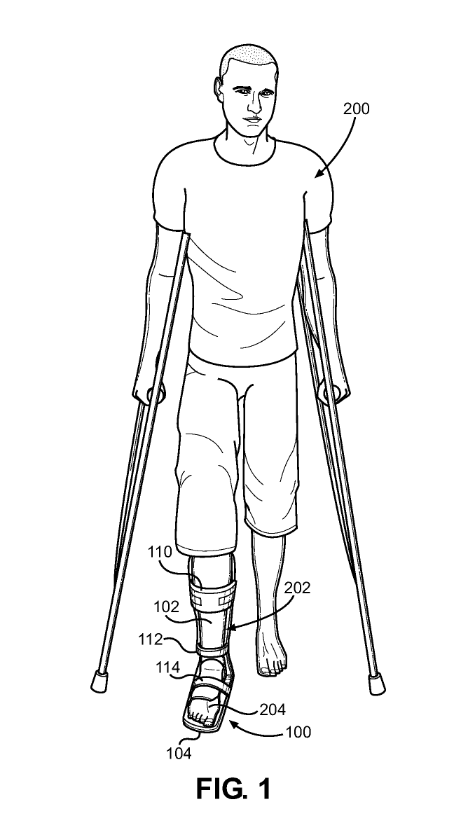

Referring now to FIG. 1, there is shown an embodiment of a leg brace device secured to the leg of a user. The leg brace device ∫100 has multiple calf straps 110, 112, and a foot strap 114 that removably secure the calf 202 and foot 204 of a user to the brace. The body of the brace is an upright support 102 and a foot support 104, joined to form an approximately 90-degree angle. Calf straps 110 and 112 encircle the upper and lower calf 202. In an embodiment, the straps may cinch to tighten the upright support 102 to the calf 202. Foot strap 114 may have a protective pad 116 attached to, or integrated with the foot strap 114. The protective pad 116 and foot strap 114 may encircle the user's 204 and removable secure to the foot support 104. In an embodiment, the protective pad 16 may extend lengthwise for all or part of the upper surface length of the user's foot 204.

An embodiment of the leg back device provides a means for stabilizing and restraining movement of an injured limb. The leg brace device 100 may be removably secured to the calf 202 and foot 204 of a user, without the need for additional tools. In an embodiment, a user may choose to utilize crutches while wearing the leg brace and may suspend the leg forward in a manner commensurate with normal use of crutches. Alternatively, the user may deploy the retractable wheels (not shown) of the present invention and place the foot 204 secured in the leg brace device 100 on the ground such that the wheels are in contact with the ground surface. The leg can then be rolled along the ground via the wheels of the leg brace device. Crutches may be employed for additional stability, or to reduce the weight placed on the injured limb.

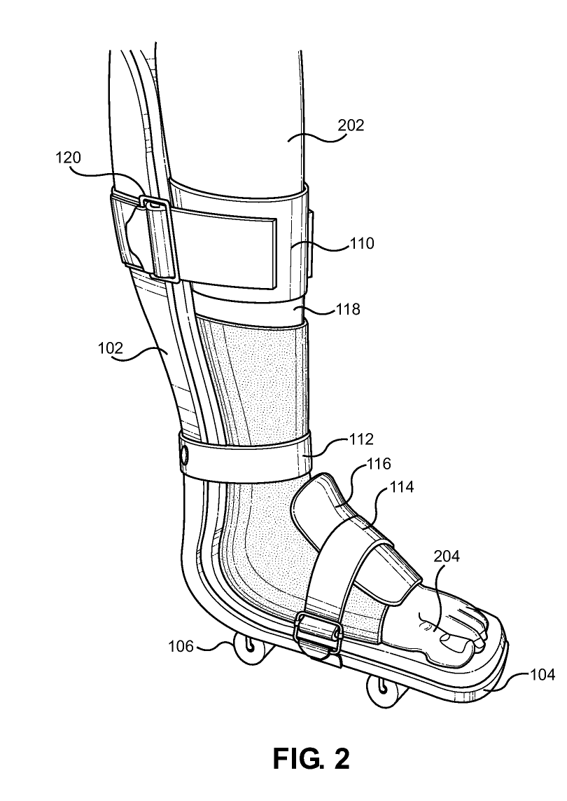

FIG. 2 illustrates a side view of an embodiment of a leg brace device with a user's foot and calf removably secured to the device. The upright support 102 and foot support 104 of the leg brace device 100 may be joined such that the upright support provides a substantially vertical support member that rests along the back of a user's calf 202 when secured, and the foot support 104 provides a lateral support member that may rest under and support the sole of a user's foot 204 when in use. In some embodiments, the position of the upright support 102 with respect to the foot support 104 may be fixed during manufacture. In some embodiments, the upright support 102 may be pivotably connected to the foot support 104 either by pivot joint or by flexible material. In alternative embodiments, the upright support and/or foot support may be contoured to conform to the natural shape of a user's leg and foot, e.g. curved to provide arch support for feet or curved outward to receive the upper calf region.

Calf straps 110, and 112 and foot trap 114 extend across the front or upper portion of the calf 202 and 204 and may secure to an opposing side of the leg brace device. In some embodiments, straps 110, 112, 114 may completely encircle the appendage of a user and affix to themselves. As illustrated, the straps may have resizing buckles 120 that enable cinching or loosening of the strap. In an embodiment, straps 110, 112, 114 may connected to opposing sides of the leg brace device via snaps, buckles, hook and loop fasteners, or the like. Protective pads 116, 118 may be attached to or integrated into straps, shown in FIG. 2 as a covering for the upper foot. Alternative embodiments include protective pads 118 that cover a portion of the front of any calf strap to reduce chaffing of the user's skin and relieve pressure from the straps.

Wheels 106 are affixed to wheel mount assemblies 108 that retractably extend outward from the exterior of the foot support 104. When the wheels 106 are extended, the user may place them on the floor and gently exert forward pressure to move the injured limb forward. Retrograde motion may similarly maneuver the limb backward. In this manner the user may move the injured appendage within having to lift and suspend the limb during ambulatory motion.

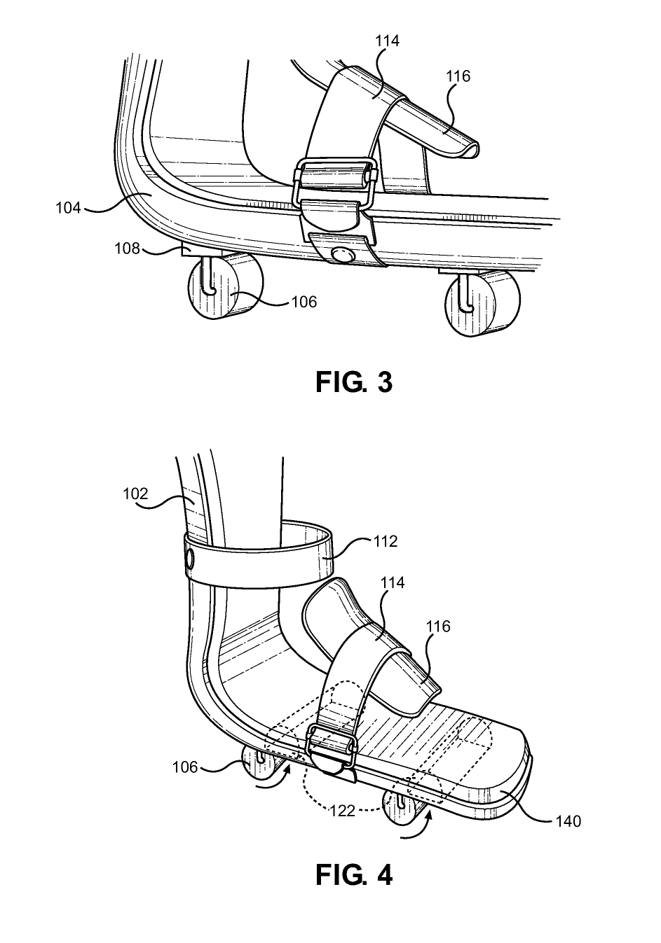

FIG. 3 illustrates an exploded side view of an embodiment of a leg brace device. Interior surfaces of the upright support 102 and foot support 104 may be formed of or covered in a soft durable material suitable of r use against a wearer's bare skin. Exterior surfaces of the leg brace device may be covered or bare of additional materials. Straps 112, and 114 may extend from one side of the upright support 102 and foot support 104 to the other, or may be encircle by same. Resizing buckles 120 may be secured to one side of the upright support 102 and/or foot support 104, enabling a strap to pass through the buckle without directly securing to the upright or foot support 102, 104. In other embodiments, the straps secure directly to the side of the upright support 102 or the foot support 104.

The wheel assemblies 108 are retractably secured to the foot support 104. By way of example, multiple wheel recesses may be disposed along and within the foot support 104, and the wheel assemblies 108 pivotably secured to the foot support such that they may pivot into the wheel recesses, bringing the wheels into the recesses. This is a stowed configuration, wherein the wheels 106 lie flush with or are recessed from the exterior surface of the foot support 104. In an embodiment, the wheel assemblies may be pivotably connected to the portion of each wheel recess proximal to the upright support 102. In another embodiment, the wheel assemblies may be pivotably connected to portions of the wheel recesses proximal to the lateral edges of the foot support. The construction of wheel assemblies for the purpose of holding a wheel and enabling rotational motion thereof, is known it he art of simple mechanics and as such, specific structure of the wheel assemblies is not addressed herein.

FIG. 4 illustrates a perspective view of an embodiment of a leg brace device. The body of the device 100 is formed by an upright support 102 connected to one end of a foot support 104. Straps 110, 112, and 114 extend across the supports 102, 104 and are resizeable to facilitate removable securement of a user's calf and foot. Wheels 106 retractably extend away from the exterior surface of the of the foot support 104 such that the wheel are able to stay in a vertically aligned deployed configuration when weight is placed thereon, and are able to retract into wheel recesses 122 when rotational pressure is applied thereto.

Protective pads 116 may be attached to or integral to the straps. These pads may be made of soft material, layers of padding, quilted material, foam, or other pliable deformable material. Presence of protective pads facilitates dispersion of pressure along the affected area, and may alleviate some of the discomfort associated with the cinching of straps. In another embodiment, the protective pad may be rigid, with a padded interior surface. Rigid pads may help hold the wear's limb in position, and may be useful for persons wearing casts along with the leg brace device. In some embodiments, all straps have protective pads. In other embodiments, only the foot support has a protective pad 116.

In various embodiments, a leg brace is provided that removably secures to the calf and foot areas of a user. Strapping the limb to the leg brace device via a series of straps, restricts the movement of the limb thereby reducing the risk of further injury. Retractable wheels enable the user to roll the limb rather than suspending it, or dragging it along. This may alleviate some of the pain associated with ambulatory motion using injured legs. If the user desires to use the leg brace without the wheels, he or she may retract the wheels into recesses.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A leg brace device, comprising:
an upright support; and
a foot support joined at one end to said upright support to form an L-shaped body; and
a plurality of straps connected to said upright support and said foot support; and
a plurality of wheels retractably connected to said foot support, wherein said wheels may be retracted within wheel recesses disposed along and within said foot support.

2) The leg brace device of claim 1, wherein said upright support is substantially vertical in orientation.

3) The leg brace device of claim 1, wherein said foot support is substantially horizontal in orientation.

4) The leg brace device of claim 1, further comprising one or more protective pads attached to said straps.

5) The leg brace device of claim 4, wherein at least one of said one or more protective pads are cushioned.

6) The leg brace device of claim 4, wherein at least one of said one or more protective pads are rigid.

7) The leg brace device of claim 1, wherein said wheels are connected to said foot support via wheel assemblies.

8) The leg brace device of claim 7, wherein said wheel assemblies are pivotably connected to said foot support.

9) The leg brace device of claim 1, wherein said wheel lie flush with, or recessed from said exterior surface of said foot support, when stowed within said wheel recesses.

10) The leg brace device of claim 1, wherein said body is contoured to conform to the shape of a human calf and foot.
